(19) United States
(12) Patent Application Publication
(10) Pub. No.: US 2002/0168432 A1
(43) Pub. Date: Nov. 14, 2002

(54) Use of fermented soy extract in inhibiting vancomycin-resistant enterococci

(76) Inventor: Kung-Ming Lu, Taipei (TW)

Correspondence Address: LARIVIERE, GRUBMAN & PAYNE, LLP, 1 LOWER RAGSDALE, BLDG. 1, SUITE 130, P.O. BOX 3140, MONTEREY, CA, 93942, US

(21) Appl. No.: 09/884,162

(22) Filed: Jun. 20, 2001

Related U.S. Application Data
(63) Continuation-in-part of application No. 09/812,579, filed on Mar. 21, 2001.

(51) Int. Cl. A61K 35/78; C12P 1/00

(52) U.S. Cl. 424/757; 435/41

(57) ABSTRACT

The present invention relates to a new use of a fermented soy extracts in preventing VRE infections.

### BACKGROUND OF THE INVENTION

1. Field of the Invention

The present invention relates to a use of a fermented soy extract in controlling vancomycin resistant microorganisms.

2. Description of the Prior Art

Antibiotics are chemical compounds used to kill or inhibit the growth of infectious organisms. Currently, the term “antibiotics” now includes synthetics and semi-synthetic organic compounds. The increased use of antibiotic therapy has been accompanied with a corresponding increase in the evolution of bacterial defenses against the drugs. Now, antibiotic resistance in bacteria has reached a crisis point in healthcare, with the discovery of many bacterial isolates which display multi-drug resistance to many of the known antimicrobials. It is desirable to find one or more replacements for antibiotics at present to solve the above-mentioned problem.

Enterococci, which can cause meningitis, heart inflammation, and stomach infections, most often affects the elderly and those with a weakened immune system, are naturally resistant to a large of antibiotic including cephalosporin, aztreonam and the β-lactamase-resistant penicillins such as oxacillin. There are at least 17 different species of Enterococci, including E. avium, E. casseliflavus, E. durans, E. faecalis and E. faecium are clinically significant pathogens in humans. About 90% of enterococcal infectious are caused by E. faecalis and 7% by E. faecium. In particular, beginning in the late 1980s, the isolates of vancomycin-resistant Enterococci (VRE) spread rapidly in major hospitals throughout the USA. Further studies of community transmission of VRE both within and between animal and human populations are urgently needed.

The development of the drugs for inhibiting VRE is extremely desirable. U.S. Pat. No. 6,037,447 discloses certain glycopeptide dimmers for use in inhibiting VRE in which two glycopeptide units are covalently linked to one another through their disaccharide amine, via a linking radical. U.S. Pat. No. 5,989,542 is directed to polysaccharides that can be used to induce the production of antibodies to specific strains of enterococcal bacteria. U.S. Pat. No. 5,861,157 provides a purified bacterial protein expressed during infection due to Streptococci or Enterococci and isolated from human sera, together with immunogenic fragments, analogs, inhibitors, antibodies and antigenic fragments specific thereto.

Up to now, the VRE still cannot be effectively controlled. Therefore, there is a need to development a medicament for controlling VRE infections.

### SUMMARY OF THE INVENTION

One aspect of the invention is to provide a composition for use in the manufacture of the medicament for treating Vancomycin Resistant Enterococci (VRE) infections, comprising the fermented soy extract which is made with the fermentation of an aqueous soy extract with at least one lactic acid bacteria.

The other aspect of the invention is to provide a method for inhibiting Vancomycin Resistant Enterococci (VRE) in a subject comprising administering an effective amount of a fermented soy extract to the subject, wherein the fermented soy extract is made with the fermentation of an aqueous soy extract with at least one lactic acid bacteria.

### DETAILED DESCRIPTION OF THE INVENTION

Process for Producing the Fermented Soy Extract

According to the invention, the fermented soy extract is made by fermentation of an aqueous soy bean extract with at least one lactic acid bacteria, e.g. a strain of one Lactobacillus species, followed by sterilization, e.g. by heat, of the fermented liquid with optional filtration and concentration. In addition to using at least one lactic acid bacteria, the fermentation of the aqueous soy extract can be conducted with at least one yeast such as a Saccharomyces species, e.g. Saccharomyces cerevisiae. The fermentation of the aqueous soy extract with one or more lactic acid bacteria and the optional yeast or yeasts can be carried out sequentially in any order or simultaneously, preferably simultaneously.

The fermented soy extract is produced by fermentation of soy bean extract with at least one lactic acid bacteria, e.g. one or more strains of a Lactobacillus species or several strains of a number of Lactobacillus species, optionally together with at least one yeast, e.g. a strain of a Saccharomyces species. If more than one microbe is used in the fermentation, the fermentation can be conducted with the microbes sequentially or simultaneously. Preferably, an aqueous extract of non-genetically modified organic soybeans of selected grade is used as a starting material. Preferably, the fermentation is carried out using a heterogeneous culture of Lactobacillus, for example, a culture of 5, 10, 15, 20, 25 or 30 strains of Lactobacillus. More preferably, at least one yeast is added to the heterogeneous culture of Lactobacillus. The strains of Lactobacillus that can be used include, for examples, Lactobacillus acidophilus CCRC 10695, 14026, 14064, 14065 and/or 14079, Lactobacillus delbrueckii bulgaricus CCRC 10696, 14007, 14009, 14010, 14069, 14071, 14098 and/or 16054, Lactobacillus lactis CCRC 10791, 12267, 12306, 12312, 12315, 12323, 14016, 14015 and/or 14117, Lactobacillus kefir CCRC 14011, and/or Lactobacillus kefiranofaciens CCRC 16059. The yeast that can be used include, for example, Saccharomyces cerevisiae CCRC 20577, 20578, 20581, 21494, 21550, 21797, 21805, 22138, 22234, 22337, 22731 and/or 22728, and/or Candida kefyr CCRC 21269, 21742 and/or 22057. After fermentation, the fermented liquid is sterilized, e.g. by heat or irradiation, preferably by heat, to obtain a sterilized liquid. Preferably, the sterilized liquid is filtered or centrifuged, preferably filtered, to remove most or all of the dead microbes to obtain the fermented soy extract. More preferably, the filtration step is followed by removal of some of the water from the filtrate to concentrate the fermented liquid to obtain the fermented soy extract. Unless otherwise specified, the tests performed in this application involved the fermented soy extract after the concentration step. Optionally, the fermented soy extract can be dried, e.g. via lyophilization, to obtain the fermented soy extract in a powder form.

The process can be carried out by mixing organic soybean (with fat removed) with distilled water at a ratio of 1:10. The mixture is heated at 100° C. for 30 minutes and then filtered to obtain a soy extract. Beef and kelp are boiled in distilled water for 30 minutes to obtain a broth. Salt, sugar and agar are added to produce a special agar medium. The lactic acid bacteria and yeast strains are added to the special agar medium. The lactic acid bacteria with the optional inclusion of the yeast in the medium are transferred to the soy extract and incubated at 36-43° C. for 45-50 hours. Preferably, the various strains of the microbes are grouped according to similar growth characteristics, e.g. any requirements of unique nutrient medium, whether the microbial strains could produce a good smell after fermentation and whether the grouped microbes can survive in the unique condition, so that groups of the microbes are added to the soy extract separately before the incubation. The purpose of this step is to reduce any negative interaction among the various strains. Also preferably, equal proportion of the different groups of microbial strains are added to the soy extract before the incubation and the resulting extract is incubated at 40° C. for 45-47 hours. Upon completion of the incubation period, the heterogeneous culture is then transferred to the soy extract again and incubated at 36-43° C. for 100-150 hours. The final fermented extract is heat sterilized and filtered; and 95% of the water content of the filtrate is removed in a concentrator to obtain a fermented soy extract in a concentrated or condensed form. The upper layer is then filtered through porcelain, and thereafter dispensed in containers and sealed.

A fermented soy extract was prepared as described above. The specific gravity of the fermented soy extract was 1.136 g/ml with 71.49% moisture, 5.15% ash, 0.16% crude fat, 5.45% crude protein, 0.15% crude fiber and carbohydrate. It also contained several vitamins and minerals: vitamin B1, 0.004 mg/100 g; vitamin B2, 0.12 mg/100 g; iron, 2.17 mg/100 g; calcium, 113.55 mg/100 g and phosphorous, 379.19 mg/100 g.

Uses of the Fermented Soy Extract

In this invention, the fermented soy extract may be administered alone or in a composition comprising the fermented soy extract and a pharmaceutically acceptable carrier, diluent and/or excipient. The fermented soy extract may be administered at a dose of about 0.001 to 40 mil/kg body weight, with a maximum dose of 2000 ml per person per administration. Preferably, the dose of the fermented soy extract is 0.01 to 20 ml/kg, more preferably 0.1 to 5 ml/kg, body weight of the subject. These doses are based on the fermented soy extract in the concentrated form, but appropriate doses of the fermented soy extract in the unconcentrated form or dry powder form can be calculated accordingly. The dose can be adjusted based on the health condition of the subject or the disease to be prevented or treated.

The fermented soy extract was demonstrated to be highly safe for daily intake of 1-10 ml on a long-term basis in a 6 months chronic toxicity study of rodents. Mice receiving a dose of 10 ml/kg and 1 ml/kg for 28 days did not exhibit any significant difference or abnormal symptom in a subacute oral toxicity study. No signs of gross toxicity or mortality were observed in two groups of tested animals administered 20 ml/kg and 1 ml/kg in an acute oral toxicity study of rodents. The fermented soy extract was demonstrated to be non-mutagenic in Ames test, to not cause chromosomal damage in mammalian cells in vitro and to not induce micronuclei in bone marrow cells in ICR mice tested.

When the fermented soy extract is administered in pregnant women, the dosage of the fermented soy extract can be increased during pregnancy until the daily intake reaches 12 ml. The fermented soy extract can be administered at early and midstage pregnancy, as well as delivery. Results showed that the fermented soy extract could improve symptoms, including constipation, nausea, vomiting, and gastrointestinal discomfort, commonly found in pregnancy. In addition, the administration of the fermented soy extract can reduce abnormalities during pregnancy and at delivery. The fermented soy extract is not only good for health improvement during pregnancy, but it also produces no adverse effect as a long-term dietary supplement. Daily administration of the fermented soy extract to newborns or infants daily increases weight gain of the babies or infants. Similarly, increased weight gain can be achieved in infants of nursing mothers continuously taking the fermented soy extract.

The fermented soy extract can also enhance hemopoeitic and liver functions after a surgical operation as demonstrated through daily administration of 1 ml of the fermented soy extract along with other therapeutic products to women undergoing operation after hospital admission except for the surgery day and several post-surgery days.

Use as an Antimicrobial Agent to Prevent or Treat Infections

The fermented soy extract has demonstrated antimicrobial activity in vitro and in vivo. It inhibits the growth of Helicobacter pylori, ampicillin and methycillin resistant Staphylococcus aureus, Salmonella typhimurium, Bacillus subtilis, E. coli, Proteus vulgaris and Vancomycin resistant Enterococci. Preferably, the Vancomycin resistant Enterococcus is selected from the group consisting of E. avium, E. casseliflavus, E. durans, E. faecalis and E. faecium. More Preferably, the Vancomycin resistant Enterococcus is selected from the group consisting of E. avium, E. casseliflavus, E. durans and E. faecali. More Preferably, the Vancomycin resistant Enterococcus is selected from the group consisting of E. durans, E. faecalis and E. faecium. More Preferably, the Vancomycin resistant Enterococcus is selected from the group consisting of E. avium, E. faecalis and E. faecium. More Preferably, the Vancomycin resistant Enterococcus is selected from the group consisting of E. faecalis and E. faecium. Most preferably, the Vancomycin resistant Enterococcus is E. faecalis.

The effective concentration of fermented soy extract is generally in the range of 1-10%. The selective antimicrobial decontamination effect of fermented soy extract for prophylaxis of bacterial infection in patients who are under risk of developing neutropenia due to the concurrent treatment of anti-cancer chemotherapy is also demonstrated in 100 patients. Preferably, the Vancomycin resistant Enterococcus is selected from the group consisting of E. avium, E. casselifiavus, E. durans, E. faecalis and E. faecium. More Preferably, the Vancomycin resistant Enterococcus is selected from the group consisting of E. avium, E. casseliflavus, E. durans and E. faecali. More Preferably, the Vancomycin resistant Enterococcus is selected from the group consisting of E. durans, E. faecalis and E. faecium. More Preferably, the Vancomycin resistant Enterococcus is selected from the group consisting of E. avium, E. faecalis and E. faecium. More Preferably, the Vancomycin resistant Enterococcus is selected from the group consisting of E. faecalis and E. faecium. Most preferably, the Vancomycin resistant Enterococcus is E. faecalis.

Compositoin

The invention also provides a composition for use in inhibiting VRE infections, comprising the fermented soy extract which is made with the fermentation of an aqueous soy extract with at least one lactic acid bacteria. Preferably, the composition can be used in the manufacture of the medicament for treating or preventing VRE infections. Preferably, the composition also can be used as antibactericidal agents.

Within the scope of the present invention is a fermented extract of a Chinese herb prepared in a process similar to the one described above with the substitution of the soy bean with the Chinese herb. The fermented extract of the Chinese herbs can be Glycyrrhiza uralensis Fish, Lycium barbarum, Coix lacryma-jobi L var., Ma-yune Stapf, Sophora tonkinensis gapnep., Cassia btusifolia., Scutellaria baicalensis Georgi, Artemisia capillaries Thunb., Coptis chinensis Frsnch., Gentiana scabra Bge., Nelumbo nucifera Gaertn., Chrysantheiferamum morifolium Ramat., Gardenia jasminoides Ellis, Hordeum vulgare L., Cinnamomum cassia Presl, Raph, anus sativus L., Dioscorea opposita Thunb., Angelica sinensis (Oliv.), Ligusticum chuanxiong Hort., Notopterygium incisum, Paeonia lactiflora Pall., Allium satium L., Schisandra chinensis (Turcz.) Baill, Rehmannia glutinosa Libosch., Acanthopanax gracilistylus W. W. Smith, Equus asinus L., Ligustrum lucidum Ait., Phaseolus radiatus L., Triticum aestivum L., Dolichos lablab L., Atractylodes macrocephala Koidz., Saposhnikovia divaricata, Lonicera japonica Thund., Cinnamomum cassia Presl, Zingiber officinale Rosc., Gastrodia elata Bl., Asparagus cochinchinensis(Liur.)Merr., Dendrobiun loddigesii Rolfe., and Sesamum indicum L.

This invention will now be described with reference to the following non-limiting examples.

### EXAMPLE

The anti-microbial activities of the fermented soy extract were demonstrated by determining with in vitro and in vivo methods.

(A) In vitro Studies

Inhibition Zone Test

In the first experiment, Salmonella typhimurium, Bacillus subtilis, three strains (TMU-C74, TMU-D 16 and TMU-E86) of Helicobacter pylori and vancomycin resistant Enterococcus feacalis were cultured in nutrient broth or BHI broth and transferred to Mueller Hinton agar plates or chocolate agar plates. The fermented soy extract was put on a paper disk on the agar plate and the size of an inhibition zone was measured after incubation at 37° C. The data are shown in below Table 1.

TABLE 1

| Microbe | Fermented Soy Extract | Inhibition Zone (mm) |
|---|---|---|
| Salmonella typhimurium | Undiluted | 11 |
| Bacillus subtilis | Undiluted | 14 |
| H. pylori TMU-C74 | Undiluted | 15 |
| H. pylori TMU-D16 | Undiluted | 16 |
| H. pylori TMU-E86 | Undiluted | 15 |
| V.R. E. feacalis | Undiluted | 25 |
| V.R. E. feacalis | Diluted 50% | 15 |

Minimal Inhibitory Concentration Test

In another experiment, the minimal inhibitory concentrations (MICs) of the fermented soy extract were determined in Salmonella typhimurium (ATCC 14028), Bacillus subtilis (CRCC 10447), Staphylococcus aureus (ATCC 25923), Escherichia coli (ATCC 25922), Pseudomonas aeruginosa (ATCC 27853), Proteus vugris (ATCC 13315) and vancomycin resistant Enterococcus feacalis. Suspensions of these bacteria were adjusted to 3×10⁵ CFU/ml. The adjusted bacteria suspensions were added to a 96-well plate with or without various concentrations, i.e. 10%, 5%, 2.5%, 1.25%, 0.65%, or 0.32%, of the fermented soy extract. The plate was incubated at 37° C. for 15 hours. The MICs were determined after incubation and shown in below Table 2.

TABLE 2

| | MIC of Fermented |
|---|---|
| Microbe | Soy Extract (%) |
| Salmonella typhimurium | <2.5 |
| Bacillus subtilis | ≦2.5 |
| Staphylococcus aureus | ≦2.5 |
| Escherichia coli | ≦5 |
| Pseudomonas aeruginosa | ≦2.5 |
| Proteus vugris | ≦2.5 |
| V.R. Enterococcus feacalis | ≦1.25 |

Minimal Bactericide Concentration Test

In another further experiment, the minimal bactericide concentrations (MBCs) of the fermented soy extract were determined in Salmonella typhimurium (ATCC 14028), Bacillus subtilis (CRCC 10447), Staphylococcus aureus (ATCC 25923), Escherichia coli (ATCC 25922), Pseudomonas aeruginosa (ATCC 27853), Proteus vugris (ATCC 13315) and vancomycin resistant Enterococcus feacalis. Suspensions of these bacteria were adjusted to 10⁶ CFU/ml. The adjusted bacteria suspensions were added to a 96-well plate with or without various concentrations, i.e. 10%, 5%, 2.5%, 1.25%, 0.65%, or 0.32%, of the fermented soy extract. The plate was incubated at 37° C. for 24 hours. The MBCs were determined after incubation and shown in below Table 3.

TABLE 3

| | MBC of Fermented |
|---|---|
| Microbe | Soy Extract (%) |
| Salmonella typhimurium | ≦10 |
| Bacillus subtilis | ≦2.5 |
| Staphylococcus aureus | ≦5 |
| Escherichia coli | ≦5 |
| Pseudomonas aeruginosa | ≦2.5 |
| Proteus vugris | ≦2.5 |
| V.R. Enterococcus feacalis | ≦5 |

(B) In vivo Studies

The vancomycin-resistance Entercoccus feacalis (VRE) was isolated from l patients of Hospital of Taipei Medical University. E. faecalis was grown in brain-heart infusion (BHI) broth. Semi-solid medium was prepared by adding 2% (wt/vol) agar to the diluted broth cultures (10⁸ CFU/ml) associated with 10% (wt/vol) barium sulfate. Aliquots (0.5 ml) of the final product were placed in double gelatin capsuled for peritoneal implantation.

250-300 g Sprague-Dawley rats, which were divided to three groups (two experimental groups and one control group) and housed 5 per cage in each group, were used for peritonitis model. The SD rats were purchased from the animal center of National Science Council. The SD rats were anesthetized with an intramuscular injection of ketamine (30 mg/kg of body weight). The gelatin capsule was inserted into pelvic peritoneal cavity of the rat through a midline abdominal incision. The wound was closed with a musculopertioneal layer and a skin layer by using interrupted nylon sutures.

After implantation, each rat of the two experimental groups was fed with 50% and 10% of fermented soy extract solutions respectively. At the third day, the rats were sacrificed. A midline laparotomy was performed and perimoneal fluid samples were recovered from all regions of the peritoneal cavity for bacterial counts. The bacterial titer of each group was measured as below Table 4:

TABLE 4

| Group | Titer (CFU per ml) |
|---|---|
| Control | 180 |
| 50% fermented soy extract | 40 |
| 10% fermented soy extract | 49 |

What is claimed is:

1. A method for inhibiting Vancomycin Resistant Enterococci (VRE) in a subject comprising administering an effective amount of a fermented soy extract to the subject, wherein the fermented soy extract is made with the fermentation of an aqueous soy extract with at least one lactic acid bacteria.

2. The method of claim 1, wherein the fermentation of the aqueous soy extract is conducted with at least one lactic acid bacteria and a yeast, wherein the lactic acid bacteria is a Lactobacillus species.

3. The method of claim 1, wherein the VRE is selected from the group consisting of E. aviun, E. casseliflavus, E. durans, E. faecalis and E. faecium.

4. The method of claim 1, wherein the VRE is selected from the group consisting of E. avium, E. casseliflavus, E. durans and E. faecalis.

5. The method of claim 1, wherein the VRE is selected from the group consisting of E. durans, E. faecalis and E. faecium.

6. The method of claim 1, wherein the VRE is selected from the group consisting of E. avium, E. faecalis and E. faecium.

7. The method of claim 1, wherein the VRE is selected from the group consisting of E. faecalis and E. faecium.

8. The method of claim 1, wherein the VRE is E. faecalis.

9. The method of claim 1, which can be used in the manufacture of the medicaments for treating or preventing VRE infections.

10. A composition for use in inhibiting VRE, comprising the fermented soy extract which is made with the fermentation of an aqueous soy extract with at least one lactic acid bacteria.

11. The composition of claim 10, wherein the fermentation of the aqueous soy extract is conducted with at least one lactic acid bacteria and a yeast, wherein the lactic acid bacteria is a Lactobacillus species.

12. The composition of claim 10, wherein the VRE is selected from the group consisting of E. avium, E. casselifiavus, E. durans, E. faecalis and E. faecium.

13. The composition of claim 10, wherein the VRE is selected from the group consisting of E. avium, E. casseliflavus, E. durans and E. faecalis.

14. The composition of claim 10, wherein the VRE is selected from the group consisting of E. durans, E. faecalis and E. faecium.

15. The composition of claim 10, wherein the VRE is selected from the group consisting of E. avium, E. faecalis and E. faecium.

16. The composition of claim 10, wherein the VRE is selected from the group consisting of E. faecalis and E. faecium.

17. The composition of claim 10, wherein the VRE is E. faecalis.

18. The composition of claim 10, which can be used in the manufacture of the medicament for treating or preventing VRE infections.
